(19) United States
(12) Patent Application Publication
(10) Pub. No.: US 2019/0350483 A1
(43) Pub. Date: Nov. 21, 2019

(54) Wireless Electrode Device

(71) Applicant: Mark D. Noar, Owings Mills, MD (US)

(72) Inventor: Mark D. Noar, Owings Mills, MD (US)

(21) Appl. No.: 16/417,940

(22) Filed: May 21, 2019

Related U.S. Application Data
(60) Provisional application No. 62/674,116, filed on May 21, 2018.

(51) Int. Cl. A61B 5/0492; A61B 5/08; A61B 5/00

(52) U.S. Cl. CPC A61B 5/0492; A61B 2560/0468; A61B 5/0015; A61B 5/08

(57) ABSTRACT

A wireless sensor device includes a housing. A sensor is disposed in the housing and is constructed and arranged to obtain an analog electrical signal from a human patient. A processor circuit is disposed in the housing and is constructed and arranged to convert the analog electrical signal to a digitized electrical signal. A transmitter is disposed in the housing and is constructed and arranged to transmit, in a wireless manner, data relating to the digitized electrical signal. A power supply powers the device. The sensor is preferably an electrode or a respiratory sensor.

### BACKGROUND

U.S. Pat. No. 7,160,254, hereby incorporated by reference in its entirety herein, discloses an Electroviserogram (EVG) system and method to gather and evaluate myoelectric signals from intra-abdominal organs and other motility based organs. The EVG system includes a plurality of conventional electrodes positioned on the body to obtain signals relating to myoelectrical activity from a hollow internal bodily organ. The electrodes are connected in a wired manner to a signal processing module that communicates with a computer.

Although the EVG system is well-suited for its intended purpose, there is a need for wireless electrode device that can obtain and transmit data to a remote device.

### SUMMARY

An objective of the embodiment is to fulfill the need referred to above. In accordance with an aspect of an embodiment, a wireless electrode device includes a housing, with an electrode in the housing and constructed and arranged to obtain an analog electrical signal from a human patient. An amplifier is disposed in the housing and is constructed and arranged to amplify the analog electrical signal. Filter structure is disposed in the housing and is constructed and arranged to filter the amplified analog electrical signal. An A/D converter is disposed in the housing and is constructed and arranged to convert the amplified and filtered analog electrical signal to a digitized electrical signal. A transmitter is disposed in the housing and is constructed and arranged to transmit, in a wireless manner, data relating to the digitized electrical signal. A power supply is provided for powering the device.

In accordance with another aspect of an embodiment, a wireless sensor device includes a housing, with a sensor disposed in the housing and constructed and arranged to obtain an analog electrical signal from a human patient. A processor circuit is disposed in the housing and is constructed and arranged to convert the analog electrical signal to a digitized electrical signal. A transmitter is disposed in the housing and is constructed and arranged to transmit, in a wireless manner, data relating to the digitized electrical signal. A power supply is provided for powering the device.

In accordance with yet another aspect of an embodiment, a method of obtaining and analyzing data regarding myoelectric activity from motility based organs provides a plurality of wireless electrode devices, each having an electrode and a transmitter. Each electrode contacts an external part of a patient's body. Myoelectrical data relating to an internal organ of the patient is obtained with the electrodes. The myoelectrical data is wirelessly transmitted, via each transmitter, to a portable device that is separate and remote from the plurality of wireless electrode devices. The portable device is employed to analyze the transmitted myoelectrical data.

Other objectives, features and characteristics of the present embodiment, as well as the methods of operation and the functions of the related elements of the structure, the combination of parts and economics of manufacture will become more apparent upon consideration of the following detailed description and appended claims with reference to the accompanying drawings, all of which form a part of this specification.

### BRIEF DESCRIPTION OF THE DRAWINGS

The invention will be better understood from the following detailed description of the preferred embodiments thereof, taken in conjunction with the accompanying drawing, wherein like reference numerals refer to like parts, in which:

FIG. 1 a block diagram of a wireless electrode device in accordance with an embodiment shown in communication with a portable, handheld device.

FIG. 2 is a block of a wireless sensor device in accordance with another embodiment.

### DETAILED DESCRIPTION OF EXAMPLE EMBODIMENTS

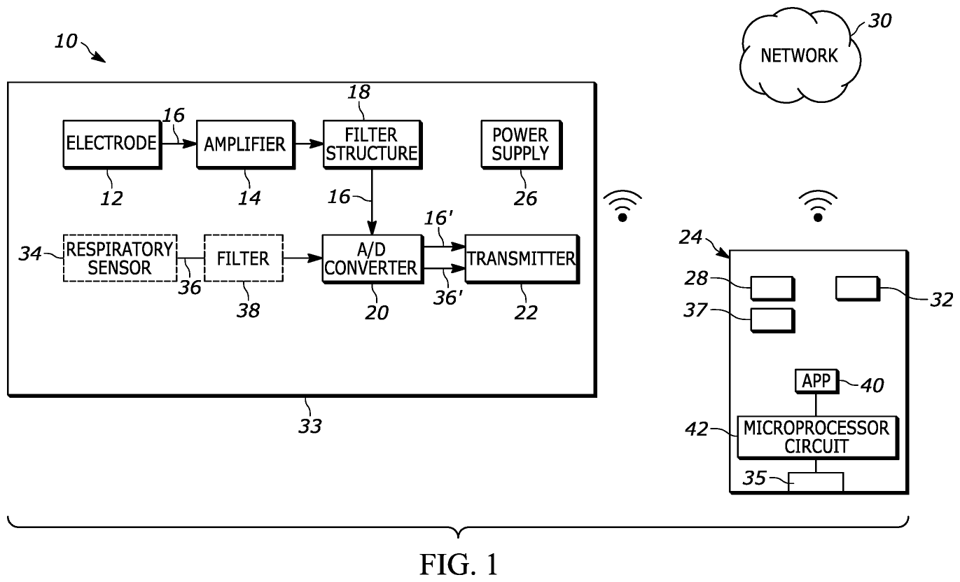

With reference to FIG. 1, a wireless electrode device is shown, generally indicated at 10, in accordance with an embodiment. The device 10 includes an electrode 12, preferably a silver-silver chloride electrode connected with an instrumentation amplifier 14 that provides the first gain stage for the electrode electrical signal 16. Filter structure 18 provides a high pass and low pass filtering of the signal 16. The electrical signal 16 is also passed to a 16-bit A/D converter 20. The digitized electrode electrical signal 16′ is then passed to a transmitter 22, which transfers data (e.g., signals 16′ and 36′) to an external portable handheld device 24 (such as a conventional smart phone or tablet) in a wireless manner. The data is thus received by a receiver 28 of the portable device 24. A power supply 26, such as a battery, powers the electrode device 10.

The portable device 24 can be considered a processing device that can communicate in a wireless manner with a network 30 via a transmitter 32 of the portable device 24. The network 30 may include at least one of a telecommunication network such as a computer network (e.g., a LAN or a WAN), the Internet, cloud-based server, and a telephone network.

The electrode device 10 can be used in any application where an electrode obtains and transmits data relating to electrical activity. It is noted that the size of the electrode device 10 and portable device 24 is not shown with regard to any scale in FIG. 1. With the miniaturization of electronics, all components of the electrode device 10 can be provided in a single housing 33 of a size similar to that of a conventional, wired-type electrode so as to be placed externally on a patient's body.

With reference to FIG. 1, for use in an Electroviscerogram (EVG) system, the device 10 can also include a respiratory sensor 34 that obtains a respiration signal 36 which is filtered by low pass filter 38 and is then passed on to the A/D converter 20. The digitized respiration signal 36′ is passed to the transmitter 22 and can be transmitted, along with the digitized electrode signal 16′, to the portable device 24. Thus, for use in obtaining myoelectric activity, a plurality of the wireless electrode devices 10 (typically three) can be employed so that a portion of the electrode 12 extends from each housing 33 and contacts an external part of a patient's body to obtain and transmit myoelectrical data to the portable device 24. Preferably, at least one of the electrode devices 10 includes the respiratory sensor 34, having a portion extending from the housing 33 so as to contact the patient, to obtain the analog respiratory signal 36. The digitized respiratory signal 36′ is received by the transmitter 22. The portable device 24 can include an application (App) 40 executed by microprocessor circuit 42 that can analyze the data (e.g., signals 16′, 36′) received from the transmitter 22 and provide a diagnosis based on the data, as disclosed in U.S. Pat. No. 7,160,254. The diagnosis results can be stored on the network 30, shared or retrieved via the network 30 or can be stored in a memory circuit 35 of the portable device 24. Also, the portable device 24 can receive data from the network 30 via receiver 28. If a location of the respiratory sensor 34 on the body is too remote from a location needed for operation of the electrode 12 in the same device 10, the electrode 12 need not be in operation so long as electrodes 12 of other devices 10 are used to obtain the myoelectrical signals.

The transmitter 22 can be in the form of a transceiver so as to also receive data from the portable device 24. For example, for an EVG application, the portable device 24 may send a calibration signal 37 to the transceiver 22 that can be received by the amplifier 14 for calibration purposes, as disclosed in U.S. Pat. No. 7,160,254.

Alternatively, instead of providing the respiratory sensor 34 and filter 38 in one of the electrode devices 10, a separate respiratory sensor device (not shown) with appropriate filter, A/D converter and transmitter can be provided that communicates in a wireless manner with the portable device 24 simultaneously with electrodes 12 of separate electrode devices 10.

Thus, by enabling the electrode device 10 to be able to transmit electrode and respiratory sensor data directly to a portable device 24, the conventional and cumbersome signal processing module, computer, printer and storage device are no longer required in an EVG system. Also, advantageously, the electrode device 10 (without the respiratory sensor 34 and filter 38) can be employed in other applications and can communicate directly with the portable device 24 having an App configured for the desired testing purpose.

The housing 33 of the electrode device 10 can be of materials suitable for implanting or embedding at least a portion thereof in a patient's tissue, so that once embedded, the device 10 can transmit data (e.g., signal 16′) to the portable device 24.

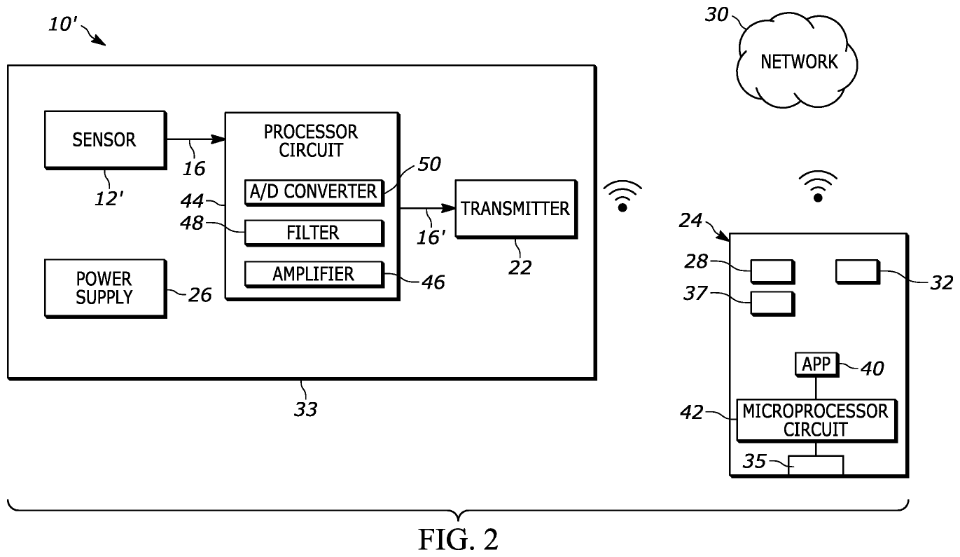

With reference to FIG. 2, a wireless sensor device is provided in accordance with another embodiment. The device 10′ includes a housing 33. A sensor 12′ is provided in the housing 33 and is constructed and arranged to obtain an analog electrical signal 16 from a human patient. The sensor 12′ can be an electrode 12 or a respiratory sensor 34 as described above. A processor circuit 44 is provided in the housing 33 and is constructed and arranged to convert the analog electrical signal 16 to a digitized electrical signal 16′. In the embodiment, the processor circuit 44 can include an amplifier circuit 46 constructed and arranged to amplify the analog electrical signal 16, a filter circuit 48 constructed and arranged to filter the amplified analog electrical signal, and an A/D converter circuit 50 constructed and arranged to convert the amplified and filtered analog electrical signal to the digitized electrical signal 16′. A transmitter 22 is provided in the housing and is constructed and arranged to transmit to the portable device 24 and/or network 30, in a wireless manner, data relating to the digitized electrical signal 16′. A power supply is provided for powering the device 10′.

The operations and algorithms described herein can be implemented as executable code within the microprocessor circuits 42, 44 as described, or stored on a standalone computer or machine readable non-transitory tangible storage medium that are completed based on execution of the code by a processor circuit implemented using one or more integrated circuits. Example implementations of the disclosed circuits include hardware logic that is implemented in a logic array such as a programmable logic array (PLA), a field programmable gate array (FPGA), or by mask programming of integrated circuits such as an application-specific integrated circuit (ASIC). Any of these circuits also can be implemented using a software-based executable resource that is executed by a corresponding internal processor circuit such as a microprocessor circuit and implemented using one or more integrated circuits, where execution of executable code stored in an internal memory circuit causes the integrated circuit(s) implementing the processor circuit to store application state variables in processor memory, creating an executable application resource (e.g., an application instance) that performs the operations of the circuit as described herein. Hence, use of the term “circuit” in this specification refers to both a hardware-based circuit implemented using one or more integrated circuits and that includes logic for performing the described operations, or a software-based circuit that includes a processor circuit (implemented using one or more integrated circuits), the processor circuit including a reserved portion of processor memory for storage of application state data and application variables that are modified by execution of the executable code by a processor circuit. The memory circuit 35 can be implemented, for example, using a non-volatile memory such as a programmable read only memory (PROM) or an EPROM, and/or a volatile memory such as a DRAM, etc.

The foregoing preferred embodiments have been shown and described for the purposes of illustrating the structural and functional principles of the present invention, as well as illustrating the methods of employing the preferred embodiments and are subject to change without departing from such principles. Therefore, this invention includes all modifications encompassed within the spirit of the following claims.

What is claimed is:

1. A wireless electrode device comprising:
a housing;
an electrode in the housing and constructed and arranged to obtain an analog electrical signal from a human patient;
an amplifier in the housing and constructed and arranged to amplify the analog electrical signal;
filter structure in the housing and constructed and arranged to filter the amplified analog electrical signal;
an A/D converter in the housing and constructed and arranged to convert the amplified and filtered analog electrical signal to a digitized electrical signal;
a transmitter in the housing and constructed and arranged to transmit, in a wireless manner, data relating to the digitized electrical signal; and
a power supply for powering the device.

2. The wireless electrode device of claim 1, in combination with a separate, portable device having a receiver and a microprocessor circuit so that the data transmitted by the transmitter can be received by the receiver, with the microprocessor circuit being constructed and arranged to execute an application that analyzes the data.

3. The wireless electrode device of claim 1, further comprising:
a respiratory sensor in the housing and having a portion constructed and arranged to contact a portion of the patient's body to obtain an analog respiratory signal; and
a filter in the housing for filtering the analog respiratory signal,
wherein the filtered analog respiratory signal is received by the A/D/converter and converted to a digitized respiratory signal, with the digitized respiratory signal being received by the transmitter.

4. The wireless electrode of claim 1, wherein a portion the electrode is constructed and arranged to contact a portion of the patient's body.

5. The wireless electrode of claim 1, wherein the housing is constructed and arranged such that at least a portion thereof can be implanted or imbedded in tissue of the patient.

6. A wireless sensor device comprising:
a housing;
a sensor in the housing and constructed and arranged to obtain an analog electrical signal from a human patient;
a processor circuit in the housing constructed and arranged to convert the analog electrical signal to a digitized electrical signal;
a transmitter in the housing and constructed and arranged to transmit, in a wireless manner, data relating to the digitized electrical signal; and
a power supply for powering the device.

7. The wireless sensor device of claim 6, wherein the processor circuit includes:
an amplifier circuit constructed and arranged to amplify the analog electrical signal;
a filter circuit constructed and arranged to filter the amplified analog electrical signal; and
an A/D converter circuit constructed and arranged to convert the amplified and filtered analog electrical signal to the digitized electrical signal.

8. The wireless sensor device of claim 6, wherein the sensor is an electrode.

9. The wireless sensor device of claim 6, wherein the sensor is a respiratory sensor.

10. The wireless sensor device of claim 6, in combination with a separate, portable device having a receiver and a microprocessor circuit so that the data transmitted by the transmitter can be received by the receiver, with the microprocessor circuit being constructed and arranged to execute an application that analyzes the data.

11. A method of obtaining and analyzing data regarding myoelectric activity from motility based organs, the method comprising:
providing a plurality of wireless electrode devices, each having an electrode and a transmitter,
contacting each electrode with an external part of a patient's body,
obtaining with the electrodes, myoelectrical data relating to an internal organ of the patient,
wirelessly transmitting, via each transmitter, the myoelectrical data to a portable device that is separate and remote from the plurality of wireless electrode devices, and
employing the portable device to analyze the transmitted myoelectrical data.

12. The method of claim 11, further comprising:
providing a wireless respiratory device including a respiratory sensor and a transmitter,
contacting the respiratory sensor with an external part of patient's body,
obtaining with the respiratory sensor, respiration data relating to patient,
wirelessly transmitting, via the transmitter of the wireless respiratory device, the respiration data to the portable device, and
employing the portable device to analyze the transmitted myoelectrical data and respiration data.

13. The method of claim 11, wherein the portable device analyzes the transmitted myoelectrical data to provide a diagnosis of the internal organ.

14. The method of claim 12, wherein the portable device analyzes the transmitted myoelectrical data and respiration data to provide a diagnosis of the internal organ.

15. The method of claim 11, wherein the wireless electrode device comprises:
a housing;
an electrode in the housing and constructed and arranged to obtain an analog electrical signal from a human patient;
an amplifier in the housing and constructed and arranged to amplify the analog electrical signal;
filter structure in the housing and constructed and arranged to filter the amplified analog electrical signal;
an A/D converter in the housing and constructed and arranged to convert the amplified and filtered analog electrical signal to a digitized electrical signal;
a transmitter in the housing and constructed and arranged to transmit, in a wireless manner, data relating to the digitized electrical signal; and
a power supply for powering the device.
